(19) United States
(12) Patent Application Publication
(10) Pub. No.: US 2009/0283493 A1
(43) Pub. Date: Nov. 19, 2009

(54) TAMPER-PROOF CLOSURE

(76) Inventor: Norbert Witowski, Wolfenbüttel (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/719,462

(22) PCT Filed: Nov. 15, 2005
(86) PCT No.: PCT/DE05/02075

§ 371 Date: Jul. 7, 2009

(30) Foreign Application Priority Data
Nov. 17, 2004 (DE) 10 2004 055 453.6

(51) Int. Cl. B65D 49/12; B65D 55/02; A61M 5/00

(52) U.S. Cl. 215/258; 215/216; 604/111

(57) ABSTRACT

The invention relates to a tamper-proof closure for a medical syringe, comprising a cover cap (7) and a fixing ring (8) for fixing the cap onto the needle base (6) of the syringe cylinder(2). The cover cap (7) and the fixing ring (8) are joined by connecting pieces (14). First projections (16) extend from the cover cap (7) to the fixing ring (8). Second projections (17) extend from the fixing ring (8) to the cover cap (7).

The present invention relates to a tamper-proof closure for a medical syringe. A syringe of this type is known from DE 195 37 163 C1.

The known syringe has a syringe cylinder with a needle base. A covering cap serves to cover the needle base. The covering cap is connected to a fixing ring at a distance therefrom by webs. The covering cap together with the fixing ring is pushed onto the needle base of the syringe cylinder such that the fixing ring is firmly attached to the syringe cylinder. So that the webs are not destroyed during the pushing-on operation, projections which maintain the distance between the covering cap and the fixing ring are provided either on the covering cap or on the fixing ring.

However, a tamper-proof closure of this type can only be pushed on in the longitudinal axis of the covering cap, which longitudinal axis coincides with the longitudinal axis of the syringe cylinder. Should it be attempted to rotate the covering cap in order possibly to be able to carry out a screwing movement, shearing forces are exerted on the webs and lead to the webs being torn off. In actual fact, the covering cap is removed by tearing the webs.

A syringe and a tip cap for it are revealed in DE 199 56 243 A1. The tip cap has an inner cap which can be placed onto the tip of a syringe cylinder. An outer cap and a casing connected to it by webs is placed on the inner cap, with a sleeve with an internal thread being provided in between.

A plastic cap with a mechanism indicating when it has been opened is revealed in DE 695 03 825 T2, the plastic cap having a cap body comprising an upper plate and a skirt-like side wall section which extends downward from the circumferential edge of the upper plate, and the plastic cap having a circumferential band section at the lower end of the skirt-like side wall section.

A screw closure for a container, in particular canister, is provided in DE 203 07 356 U1, the screw closure having a threaded cap with a closed and an open end. A circular tamper-proof ring 20 has projections and is connected to the threaded cap via tear-off segments.

A child-proof and tamper-proof container closure is revealed in DE 298 07 243 U1₁ which container closure can be screwed on and unscrewed with a tool, the tool being insertable into an opening of the container closure.

It is therefore the object of the present invention to provide a tamper-proof closure of the type described at the beginning, in which not only is it possible to push it on in the direction of the longitudinal axis of the covering cap, but also a rotational movement can be carried out without destroying it.

This object is achieved by a tamper-proof closure for a medical syringe as claimed in claim 1.

Preferred refinements of the invention are indicated in the subclaims.

By means of the provision of the first and second projections on the covering cap and on the fixing ring, respectively, a rotational movement of the covering cap is transmitted to the fixing ring, and therefore shearing forces do not have to be absorbed by the webs. The fixing ring can therefore be screwed onto the needle base of the syringe cylinder even if there is a close snug fit.

The rotational force is preferably transmitted from the covering cap to the fixing ring when those edges of the projection which face one another extend in the direction of the longitudinal axis of the covering cap.

Those edges of the projections which do not face one another are preferably provided in an inclined manner such that, by means of a reverse rotation of the covering cap, the covering cap is lifted off from the fixing ring and tears the webs. The covering cap can thus be removed without the fixing ring being detached from the needle base of the syringe cylinder. It can be seen by way of the remaining fixing ring that the covering cap has been removed. This makes it certain that a user can always see whether a covering cap has been removed.

Further features and advantages of the invention emerge from the description below of an exemplary embodiment with reference to the figures. Of the figures:

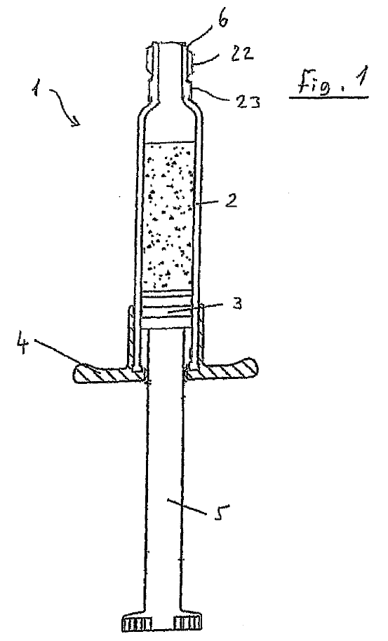

FIG. 1 shows a schematic view of a medical syringe;

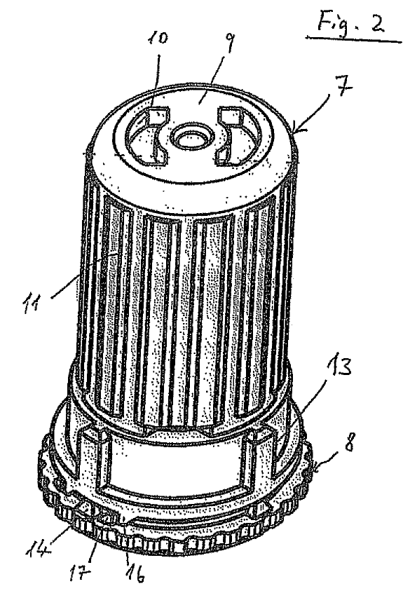

FIG. 2 shows a perspective view of an embodiment of the tamper-proof closure according to the invention;

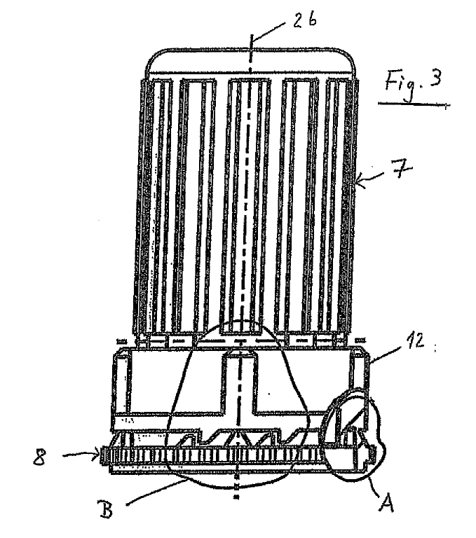

FIG. 3 shows a side view of the embodiment of the tamper-proof closure;

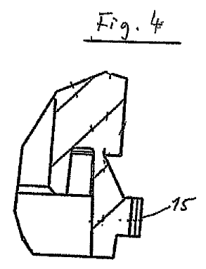

FIG. 4 shows an enlarged view of the region referred to by A in FIG. 3;

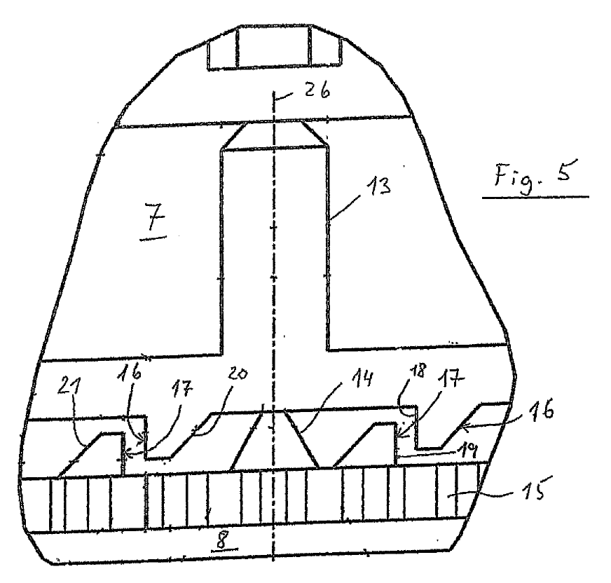

FIG. 5 shows an enlarged view of the region referred to by B in FIG. 3;

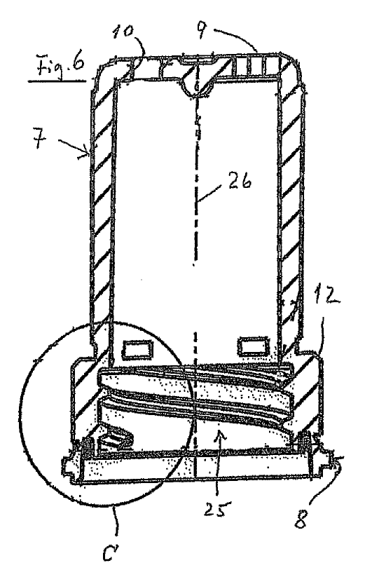

FIG. 6 shows a sectional view of the embodiment of the tamper-proof closure;

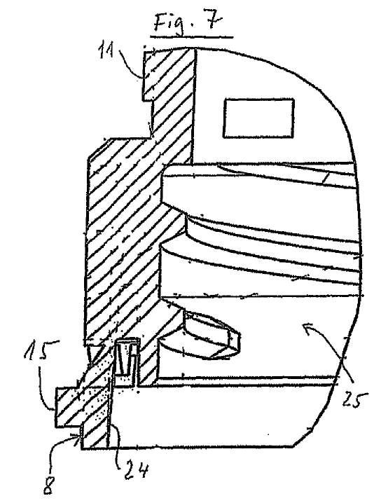

FIG. 7 shows an enlarged view of the region referred to by C in FIG. 6;

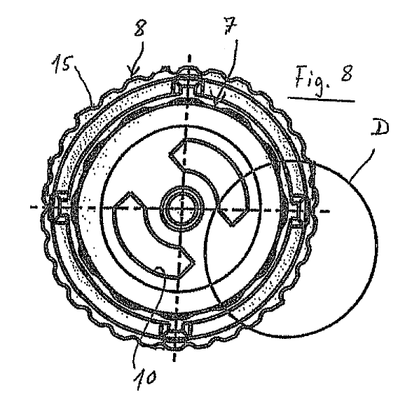

FIG. 8 shows a plan view of the embodiment of the tamper-proof closure; and

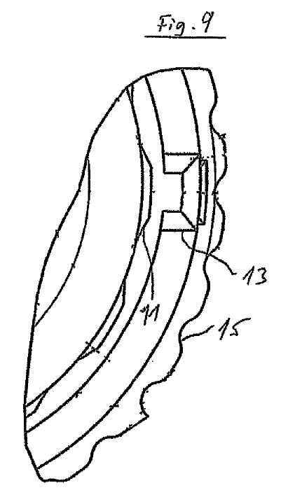

FIG. 9 shows an enlarged view of the region referred to by D in FIG. 8.

In the description below, the medical syringe 1 shown in FIG. 1 is used in the position shown there. The medical syringe 1 has a syringe cylinder 2, a plunger 3 inserted therein, a finger holder 4 and a plunger rod 5 screwed into the plunger 3. The side from which the plunger with the plunger rod is inserted is referred to as the bottom. The opposite side is referred to by top. At the top of the syringe cylinder 2, a needle base 6 is provided. The needle base 6 is explained in detail below.

The actual tamper-proof closure essentially comprises a covering cap 7 and a fixing ring 8.

As can be seen particularly in FIG. 6, the covering cap 7 is in the shape of a cup which is downwardly open. The covering cap 7 is formed in an essentially rotationally symmetrical manner about its longitudinal axis 26. On the upper side opposite the open end of the covering cap 7, a covering plate 9 is formed integrally with the covering plate 7. The covering plate 9 has slots 10, essentially in the form of arcs of a circle, for the insertion of a turning tool, as is described further on. The circumferential side surface of the covering cap 7 is provided with flutes 11 to mechanically stiffen it.

As can best be seen in FIGS. 6 and 7, the covering cap 7 is expanded at its lower end into a screwing section 12. An internal thread 25, which can be seen in particular in FIG. 7, is provided in the expanded screwing section 12. Reinforcing strips 13 are likewise 9 provided on the outer circumferential surface of the expanded screwing section 12.

As can best be seen in FIGS. 3, 4 and 5, the fixing ring 8 is connected to the lower side of the covering cap 7 by webs 14. In this embodiment, the webs 14 have a trapezoidal shape which is formed symmetrically with respect to a longitudinal axis of the covering plate. The fixing ring 8 furthermore has a ribbing 15 which surrounds the fixing ring 8 circumferentially. As can be seen in particular in FIG. 5, first projections 16 are formed on the lower edge of the covering cap 7 while second projections 17 are provided on the upper side of the fixing ring 15.

As can be seen in particular in FIG. 5, a first projection 16 of the lower side of the covering cap 7 is in each case formed adjacent to a second projection 17 on the upper side of the fixing ring 8. That edge 18 of the first projection 16 which points toward the second projection 17 extends essentially in the direction of the longitudinal axis 26 of the covering cap 7. That edge 19 of the second projection 17 which points toward the first projection 16 extends essentially in the direction of the longitudinal axis of the fixing ring 8, with the longitudinal axis of the fixing ring 8 coinciding with the longitudinal axis 26 of the covering cap 7. By contrast, that edge of the first projection 16 which faces away from the second projection 17 is inclined in relation to the longitudinal axis 26 of the covering cap 7, as can be seen in particular in FIG. 5. That edge 21 of the second projections 17 which faces away from the first projection 16 is inclined in relation to the longitudinal axis of the fixing ring 8, as shown in FIG. 5.

To use the tamper-proof closure, the tamper-proof closure is placed onto the needle base 6 of the medical syringe 1. For this purpose, the needle base 6 has an external thread 22 which is formed in a complementary manner to the internal thread 25 of the covering cap 7. Furthermore, the needle base 6 has a circumferential surface 23 which is formed in a complementary manner to an encircling inner surface 24 of the fixing ring 8. As can be seen in particular in FIG. 7, the encircling inner surface 24 of the fixing ring 8 is not precisely aligned with the longitudinal axis of the fixing ring 8. The encircling inner surface 24 of the fixing ring 8 is of conical design such that the fixing ring 8 can be placed effectively onto the outer encircling circumferential surface 23 of the needle base 6. The outer encircling surface 23 of the needle base 6 is preferably also formed conically in a complementary manner to the inner encircling surface 24 of the fixing ring 8.

The use of the tamper-proof closure takes place in the following manner. The syringe shown in FIG. 1 is provided with the plunger 3 and the plunger rod 5. The syringe is filled. A closure stopper (not shown) is inserted into the needle base 6 in order to seal the syringe 1. The tamper-proof closure, which comprises the covering cap 7 and the fixing ring 8, is then screwed onto the needle base 6 of the syringe 2 such that the internal thread of the covering cap 7 comes into engagement with the external thread 22 of the needle base 2. The covering cap 7 is screwed onto the needle base 6 until the encircling inner surface of the fixing ring 8 comes into engagement with the encircling outer surface of the needle base 6 and produces a firm connection. The screwing-on operation is facilitated by the straight edge 18 of each first projection 16 of the covering cap 7 butting against the straight edge 19 of each second projection 17 of the fixing ring 8 and carrying along the fixing ring 8. This ensures that shearing forces are not exerted on the webs 14 used for connecting the covering cap 7 to the fixing ring 8. This makes it possible for the webs 14 to be able to be of relatively weak design. In the perspective view shown in FIG. 2, the screwing movement is carried out in the clockwise direction. In FIG. 5, the covering cap 7 therefore moves somewhat to the left relative to the fixing ring 8 such that the two edges 18 and 19 come into engagement. The presence of the covering cap 7, which is connected to the fixing ring 8 by the webs 14, indicates that the syringe 1 is undamaged. The syringe is finally optionally sterilized.

If the syringe is to be used, the covering cap is rotated counterclockwise in the position shown in FIG. 2. In FIG. 5, the covering cap 7 therefore moves relatively to the right relative to the fixing ring 8, which is in frictional connection by means of its encircling inner surface 24 with the encircling outer surface 23 of the syringe 1. The movement of the covering cap 7 relative to the fixing ring 8 is carried out until the webs 14 tear. The covering cap 7 can then be rotated freely in the thread. Owing to the oblique edges 20 of the first projection 16 and the oblique edges 21 of the second projection 17, the first projection 16 can slide away over the second projection 17 such that the rotation of the covering cap 7 relative to the fixing ring 8 is made possible and the covering cap 7 can be unscrewed from the needle base 6.

The presence of the fixing ring 8 on the needle base 6 indicates that the covering cap 7 has been unscrewed and the tamper-proof closure destroyed.

### TAMPER-PROOF CLOSURE: REFERENCE NUMBERS

| Character | Feature |
|---|---|
| 1 | Medical syringe |
| 2 | Syringe cylinder |
| 3 | Plunger |
| 4 | Finger holder |
| 5 | Plunger rod |
| 6 | Needle base |
| 7 | Covering cap |
| 8 | Fixing ring |
| 9 | Covering plate |
| 10 | Slots in the shape of circular arcs |
| 11 | Flutes of the circumferential surface of the |
| | covering cap 7 |
| 12 | Expended screwing section |
| 13 | Reinforcing strips |
| 14 | Webs used for connecting the covering cap 7 |
| | to the fixing ring 8 |
| 15 | Ribbing on the fixing ring 8 |
| 16 | First projections on the lower side of the |
| | covering cap 7 |
| 17 | Second projections on the upper side of the |
| | fixing ring 8 |
| 18 | Straight edge of the first projection 16, |
| | which edge faces the second projection 17 |
| 19 | Straight edge of the second projection 17, |
| | which edge faces the first projection 16 |
| 20 | Edge of the first projection 16, which edge |
| | faces away from the second projection 17 |
| 21 | Edge of the second projection 17, which edge |
| | faces away from the first projection 16 |
| 22 | External thread of the needle base 6 |
| 23 | Encircling outer surface of the needle base 6 |
| 24 | Encircling inner surface of the fixing ring 8 |
| 25 | Internal thread of the covering cap 7 |
| 26 | Longitudinal axis of the covering cap 7 |

1. A tamper-proof closure for a medical syringe (1), with a covering cap (7) for covering the needle base (6) of a syringe cylinder (2) of the syringe (1), the covering cap (7) having an upper covering plate (9), and a fixing ring (8) for fixing said cap to the needle base (6) of the syringe cylinder (2), the covering cap (7) and the fixing ring (8) being connected to each other at a distance from each other by means of webs (14), first projections (16) extending to the fixing ring (8) being provided on the covering cap (7), and second projections (17) extending to the covering cap (7) being provided on the fixing ring (8) adjacent to the first projections (16).

2. The tamper-proof closure as claimed in claim 1, in which the webs (14) are inclined in relation to the direction of a longitudinal axis of the covering cap (7).

3. The tamper-proof closure as claimed in claim 1, in which the webs (14) have a trapezoidal shape symmetrical to the longitudinal axis of the covering cap (7).

4. The tamper-proof closure as claimed in claim 1, in which that edge of a projection (16, 17) which faces the adjacent projection (17, 16) extends essentially in the direction of the longitudinal axis of the covering cap (7), and that edge of a projection (16, 17) which faces away from the adjacent projection (17, 16) is inclined in relation to the longitudinal axis of the covering cap (7).

5. The tamper-proof closure as claimed in claim 1, in which the covering cap (7) has an internal thread (25) for screwing onto an external thread (22) on the needle base (6) of the syringe cylinder (2).

6. The tamper-proof closure as claimed in claim 1, in which circular slots (10) for the insertion of a turning tool are formed in the upper covering plate (9).

7. The tamper-proof closure as claimed in claim 1, in which the needle base (6) of the syringe cylinder (2) has a circumferential surface (23) with a conical shape and the fixing ring (8) has an inner surface (24) which is complementary thereto.
